(19) United States
(12) Patent Application Publication
(10) Pub. No.: US 2009/0242656 A1
(43) Pub. Date: Oct. 1, 2009

(54) EVAPORATOR SPRAY SYSTEM FOR AUTOMOBILE CABIN AROMA ENHANCEMENT

(76) Inventors: Thomas E. Peard, Beverly Hills, MI (US); Curtis G. Vincent, Rochester Hills, MI (US)

Correspondence Address: HARNESS, DICKEY & PIERCE, P.L.C., P.O. BOX 828, BLOOMFIELD HILLS, MI, 48303, US

(21) Appl. No.: 12/055,552

(22) Filed: Mar. 26, 2008

(51) Int. Cl. A61L 9/14; B60H 3/00

(52) U.S. Cl. 239/8; 165/138

(57) ABSTRACT

A spray nozzle or other dispensing opening is configured to deliver an aroma enhancement fluid onto an evaporator core of the HVAC system of an automobile. A fluid reservoir is configured to house the aroma enhancement fluid. Tubing can provide a flow path from the fluid reservoir to the spray nozzle. A pump unit can be located along the flow path to pump fluid from the fluid reservoir to the spray nozzle. A controller intermittently actuates the pump unit, thereby spraying the aroma enhancement fluid onto the evaporator core through the spray nozzles. In an associated method the user can select from one of a plurality of available aroma enhancing fluids. The aroma enhancement fluid can include an antibacterial component, an anti-fungal component, a deodorizer component, a fragrance component, and combinations thereof.

### FIELD

The present disclosure relates to HVAC systems for automobiles, and more particularly, to malodors that may become associated with such systems.

### BACKGROUND

The statements in this section merely provide background information related to the present disclosure and may not constitute prior art.

A pleasant aroma in an automobile cabin is highly desirable. People often refer quite favorably to that new car smell. Over time, that new car smell fades. In addition, malodors, including cigarette smoke, sweat and exhaust fumes, can be introduced into the cabin. Over time, such malodors can accumulate in the HVAC system of an automobile; and specifically on the heat evaporator core. In addition, mold bacteria or other growths can occur on at least the evaporator core, due to the moisture associated therewith.

Resin coatings can be applied to the evaporator core of an automobile HVAC system to minimize the collection of these malodors. The coating can provide a surface that resists the collection of odors. Alternatively, a deodorization filter can be installed upstream of the evaporator core to limit malodorous particles from contacting the surfaces of these components.

### SUMMARY

An automobile cabin aroma enhancement system including an evaporator core is disclosed. A dispensing opening is configured to deliver an aroma enhancement fluid onto the evaporator core. A fluid reservoir is configured to house the aroma enhancement fluid. Tubing is configured to provide a flow path from the fluid reservoir to the dispensing opening. A dispensing system is associated with the flow path and configured to deliver the aroma enhancement fluid to the delivery opening, upon actuation thereof. A controller is configured to actuate the dispensing system, thereby delivering the aroma enhancement fluid onto the evaporator core through the dispensing opening.

An automobile cabin aroma enhancement system including an evaporator core is disclosed. A spray nozzle is configured to spray an aroma enhancement fluid onto the evaporator core. A fluid reservoir is configured to house the aroma enhancement fluid. Tubing is configured to provide a flow path from the fluid reservoir to the spray nozzle. A pump unit is located along the flow path and configured to pump fluid from the fluid reservoir to the spray nozzle, upon actuation thereof. A controller is configured to intermittently actuate the pump unit, thereby spraying the aroma enhancement fluid onto the evaporator core through the spray nozzles.

A method of enhancing the aroma of an automobile cabin is also disclosed. The method includes intermittently spraying an aroma enhancement fluid at an evaporator core.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a top plan view with a partial cross-section showing the evaporator core of an exemplary embodiment of an evaporator core spray system; and

FIG. 2 is a partial perspective view of an HVAC module incorporating the system of FIG. 1.

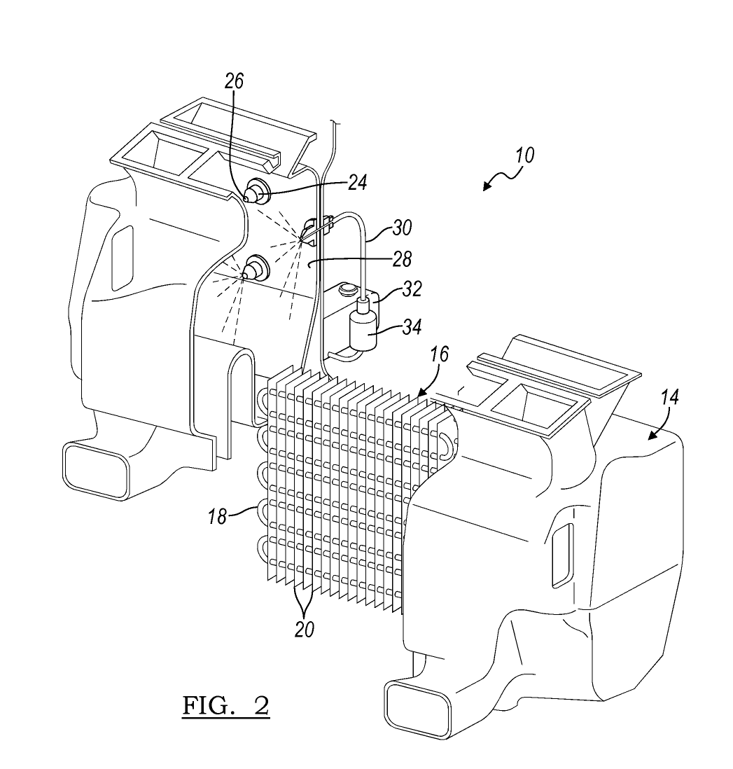

### DETAILED DESCRIPTION

The following description is merely exemplary in nature and is not intended to limit the present disclosure, application, or uses.

A heating, ventilation, and air conditioning (HVAC) module 10 can be provided within the interior of an automobile, which is adjacent the vehicle dash 12 and structure 13. The interior of the automobile also includes the passenger cabin area. The HVAC module 10 can include a module housing 14 supporting an evaporator core 16 therein. The HVAC module 10 can be a typical automobile HVAC module such as that disclosed in U.S. Pat. Nos. 5,954,578 6,311,768, 6,382,305, and 7,137,455, all of which are commonly assigned to Denso, and are hereby incorporated herein by reference.

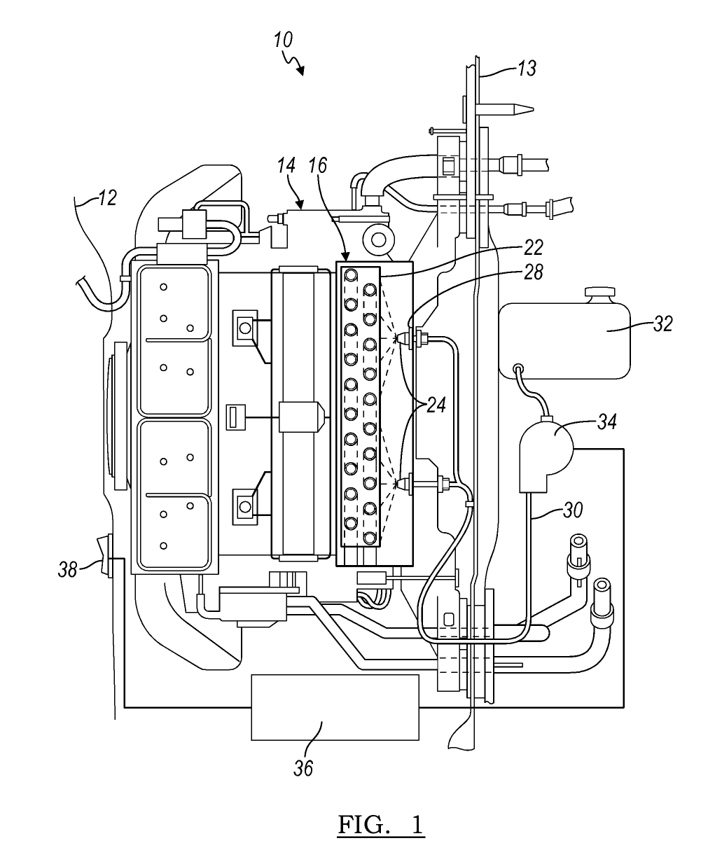

The evaporator core 16 can include a serpentine coolant flow path 18 along which a large number of extending fins 20 are located, as illustrated. Alternatively, a plate and fin evaporator core can be utilized. In either case, the fins 20 can help provide a large surface area for heat exchange between the coolant and air passing through the evaporator core 16. Thus, the evaporator core 16 can include an air flow path along which the air passes through the evaporator core 16. An air flow face area is defined, as used herein, as a cross-sectional area of the air flow path at an entry face of the evaporator core 16. This air flow path is represented by the line referenced as 22 in FIG. 1.

The module housing 14 also supports one or more spray nozzles 24, each including at least one dispensing opening 26. For example, the spray nozzles 24 can be located in a wall 28 of the module housing 14 or otherwise coupled to the housing wall 28. The spray nozzles 24 can be located adjacent the evaporator core 16 and configured to spray an aroma enhancement fluid on the evaporator core 16. In some embodiments, the spray nozzles 24 can be configured to spray the aroma enhancement fluid over substantially the entire air flow area 22.

Metal, rubber, plastic, or other suitable tubing 30 connects the nozzles 24 to a reservoir 32 configured to provide a fluid flow path from the reservoir 32 to each of the nozzles 24. The reservoir 32 is configured to house the aroma enhancement fluid. A dispensing system 34 which when activated delivers the aroma enhancement fluid to the nozzles 24 is associated with the fluid flow path 30. As illustrated, the dispensing system is a pump unit 34 which is provided along the fluid flow path 30 and configured to pump the aroma enhancement fluid to the spray nozzles 24 to spray the aroma enhancement fluid therefrom. The pump unit 34 can include an electric motor and a fluid pump. As an alternative, the aroma enhancement fluid can be provided in a replaceable pressurized fluid reservoir. Thus, a valve actuator can be used to open a valve of the pressurized fluid reservoir, or a valve can be located along the flow path to deliver the aroma enhancement fluid to the spray nozzles 24. In either of these valve alternatives, the valve is the dispensing system and is associated with the fluid flow path.

The aroma enhancement fluid preferably includes an anti-bacterial component. Thus, the aroma enhancement fluid can reduce odors that might potentially result from the growth of bacteria on the evaporator core 16. Potential anti-bacterial components can include ozone, TEG, bleaching agents containing hydrogen peroxide, and chlorine or hypochlorites. The suitability of these and other potential anti-bacterial components for this application is known to those skilled in the art. In addition, the aroma enhancement fluid can preferably be non-corrosive or can preferably include an anti-corrosive component.

The aroma enhancement fluid can preferably include an anti-fungal component. Thus, the aroma enhancement fluid can reduce odors that might potentially result from the growth of a fungus on the evaporator core 16. Potential anti-fungal components can include chlorine bleach, vinegar, alcohol, and trisodium phosphate. The suitability of these and other potential anti-fungal components for this application is known to those skilled in the art.

The aroma enhancement fluid can preferably include a fragrance component. Thus, the aroma enhancement fluid can impart a fragrance to air passing through the evaporator core 16. Potential fragrance components can include essential oils and aroma compounds, such as those commonly found in air fresheners. The suitability of various potential fragrance components for this application is known to those skilled in the art. For example, the fragrance components can be selected to mimic a new car smell. Since personal preference affects the perception of fragrance a manufacturer may offer a selection of fragrances from which consumers may choose.

The aroma enhancement fluid can preferably include a deodorizing component. Thus, the aroma enhancement fluid can adsorb odors that might potentially result from odors associated with the evaporator core 16. Potential deodorizing components can include adsorbents like sodium bicarb, activated charcoal, and silica gel. The suitability of these and other potential deodorizing components for this application is known to those skilled in the art. Deodorizing components can be used in combination with fragrance components are as an alternative to fragrance components. For example, a manufacturer may offer an unscented version along with a selection of fragrances from which consumers may choose.

A controller can be coupled to the dispensing system 34, which is illustrated as a pump unit. The controller can be an electronic control unit 36, a switch 38, or both as illustrated. The controller can be simply a switch 38 (thus eliminating the item referenced as 36 in the drawing) that is manually actuated and permits a user to actuate the dispensing system 34 to spray the aroma enhancement fluid on the evaporator core 16 at any time a user desires. This configuration allows the user full control over the intermittent actuation of the dispensing system 34.

The controller can alternatively (as illustrated) include an electronic control unit 36 linked to both the pump unit 34 and a switch 38. The electronic control unit 36 can provide for intermittent operation of the pump unit 34 at a predetermined periodic interval as described below. As described above, the switch 38 can be simply a manually actuated switch that permits a user to additionally actuate the dispensing system 34 to spray the aroma enhancement fluid on the evaporator core 16 at any time a user desires. The switch 38 can additionally or alternatively be a selector control switch that alters the predetermined length of the periodic interval.

As indicated above, the electronic control unit 36 can be configured to periodically actuate the pump unit 34 upon the passage of a predetermined time period. The predetermined time period can be associated with the absolute passage of time, with the passage of operating time of the automobile, with the passage of operating time of the evaporator core 16, or some combination thereof. For example, the electronic control unit 36 can include circuitry that actuates the dispensing system 34 upon the passage of an absolute time period that is from about 2 weeks to about 3 weeks. As another example, the electronic control unit 36 may include circuitry that periodically actuates the dispensing system upon a relatively long passage of absolute time, or upon a shorter passage of operating time of the evaporator core, whichever occurs first.

As should be apparent from the above, the controller can be independent of or linked to a main HVAC controller, for example, to monitor the passage of operating time of the evaporator core 16. As illustrated, the controller is linked to a main HVAC controller because it is provided as an integral component of the main HVAC electronic control unit 36. Alternatively, the controller can be provided as a separate electronic control unit 36 electronically linked to a main HVAC controller.

Additionally or alternatively, the controller can be linked to a sensor (not seen). For example, a temperature sensor linked to the electronic control unit 36 can be provided on the evaporator core 16. The electronic control unit 36 may additionally include circuitry configured to actuate the dispensing system 34 in relation to changes in temperature of the evaporator core 16. For example, the electronic control unit 36 can actuate the dispensing system 34 upon sensing a relatively rapid change in temperature of the evaporator core 16 indicative of a heating or cooling cycle. As another example, the electronic control unit 36 can actuate the dispensing system 34 upon the lapse of a predetermined number of heating and/or cooling cycles that the evaporator core 16 undergoes.

The method of enhancing the aroma of an automobile cabin should be apparent from the description of the evaporator spray system 10 provided herein. In addition, the method can include providing instructions for the filling of the fluid reservoir 32 with the aroma enhancement fluid and providing instructions for the operation of the system 10. Such instructions can include providing indicia in association with the fluid reservoir 32, the switch 38, an owners manual (not seen) and any combination thereof.

The foregoing discussion discloses and describes merely exemplary preferred arrangements of the present invention. One skilled in the art will readily recognize from such discussion, and from the accompanying drawings and claims, that various changes, modifications and variations can be made therein without departing from the spirit and scope of the invention as defined in the following claims. Further, there may be alternative preferred arrangements to those specifically described herein, and therefore, any use of “preferred” herein or the failure to mention any other specific alternative is not intended to exclude any such alternative arrangements.

What is claimed is:

1. An automobile cabin aroma enhancement system comprising:
an evaporator core;
a dispensing opening configured to deliver an aroma enhancement fluid onto the evaporator core;
a fluid reservoir configured to house the aroma enhancement fluid;
tubing configured to provide a flow path from the fluid reservoir to the dispensing opening;
a dispensing system associated with the flow path and configured to deliver the aroma enhancement fluid to the delivery opening, upon actuation thereof;
a controller configured to actuate the dispensing system, thereby delivering the aroma enhancement fluid onto the evaporator core through the dispensing opening.

2. The automobile cabin aroma enhancement system according to claim 1, wherein the controller is configured to provide for intermittent actuation of the dispensing system.

3. The automobile cabin aroma enhancement system according to claim 1, wherein the controller comprises a manually operated switch, thereby permitting the user to selectively actuate the dispensing system to provide intermittent operation thereof.

4. The automobile cabin aroma enhancement system according to claim 1, wherein the controller is further configured to intermittently operate the pump upon the lapse of a predetermined time period.

5. The automobile cabin aroma enhancement system according to claim 1, wherein the controller is further configured to intermittently operate the pump upon the lapse of a predetermined time period, and wherein the controller comprises a manually operated switch configured to alter the predetermined time period.

6. The automobile cabin aroma enhancement system according to claim 1, wherein the controller is linked to a main HVAC controller and further configured to operate the pump upon the lapse of a predetermined time period of the operation of the HVAC system.

7. The automobile cabin aroma enhancement system according to claim 1, wherein the controller is linked to a main HVAC controller and further configured to operate the pump during a period of operation of the HVAC system corresponding to a change in temperature of the evaporator core.

8. The automobile cabin aroma enhancement system according to claim 1, wherein the controller is linked to a main HVAC controller and further configured to operate the pump during a period of operation of the HVAC system corresponding to the presence of moisture on the evaporator core.

9. An automobile cabin aroma enhancement system comprising:
an evaporator core;
a spray nozzle configured to spray an aroma enhancement fluid onto the evaporator core;
a fluid reservoir configured to house the aroma enhancement fluid;
tubing configured to provide a flow path from the fluid reservoir to the spray nozzle;
a pump unit located along the flow path and configured to pump fluid from the fluid reservoir to the spray nozzle, upon actuation thereof;
a controller configured to intermittently actuate the pump unit, thereby spraying the aroma enhancement fluid onto the evaporator core through the spray nozzles.

10. An automobile cabin aroma enhancement system according to claim 9, wherein the spray nozzle comprises a plurality of spray nozzles, the evaporator core comprises an air flow face area, and the plurality of spray nozzles are configured to spray the aroma enhancement fluid over substantially the entire air flow face area of the evaporator core.

11. The automobile cabin aroma enhancement system according to claim 9, wherein the controller is further configured to intermittently operate the pump upon the lapse of a predetermined time period.

12. The automobile cabin aroma enhancement system according to claim 9, wherein the predetermined time period is selected from a time period from about 2 weeks to about 3 weeks.

13. The automobile cabin aroma enhancement system according to claim 9, further comprising the aroma enhancement fluid, wherein the aroma enhancement fluid comprises at least two components selected from an antibacterial component, an anti-fungal component, a deodorizer component, and a fragrance component.

14. The automobile cabin aroma enhancement system according to claim 9, further comprising the aroma enhancement fluid, wherein the aroma enhancement fluid comprises at least three components selected from an antibacterial component, an anti-fungal component, a deodorizer component, and a fragrance component.

15. A method of enhancing the aroma of an automobile cabin, the method comprising:
intermittently spraying an aroma enhancement fluid at an evaporator core.

16. The method of enhancing the aroma of an automobile cabin according to claim 15, further comprising;
providing a fluid reservoir configured to house the aroma enhancement fluid;
configuring a plurality of spray nozzles to direct the aroma enhancement fluid at an evaporator core;
providing a fluid flow path from the fluid reservoir to spray nozzles; and
filling the fluid reservoir with the aroma enhancement fluid.

17. The method of enhancing the aroma of an automobile cabin according to claim 15, further comprising offering a plurality of aroma enhancing fluids and allowing a user to choose one of the plurality of aroma enhancing fluids to be intermittently sprayed at the evaporator core.

18. The method of enhancing the aroma of an automobile cabin according to claim 15, further comprising providing instructions for the operation of the control system.

19. The method of enhancing the aroma of an automobile cabin according to claim 15, further comprising providing instructions for filling the fluid reservoir.

20. The method of enhancing the aroma of an automobile cabin according to claim 15, further comprising incorporating at least two components selected from an antibacterial component, an antifungal component, a deodorizer component, and a fragrance component into the aroma enhancing fluid.
